(19) United States
(12) Patent Application Publication
(10) Pub. No.: US 2011/0263822 A1
(43) Pub. Date: Oct. 27, 2011

(54) COMPOSITION FOR THE PROPHYLAXIS AND TREATMENT OF HBV INFECTIONS AND HBV-MEDIATED DISEASES

(75) Inventor: Karl Melber, Dusseldorf (DE)

(73) Assignee: Rhein Biotech Gesellschaft Fur Neue Biotechnologische Prozesse Und Produkte MB, Dusseldorf (DE)

(21) Appl. No.: 13/177,860

(22) Filed: Jul. 7, 2011

Related U.S. Application Data
(62) Division of application No. 11/365,210, filed on Feb. 28, 2006.
Application No. 11/365,210 is a continuation of application No. PCT/EP2004/009590, filed on Aug. 27, 2004.

(30) Foreign Application Priority Data
Aug. 29, 2003 (DE) DE 103 39 927.5

(51) Int. Cl. C07K 14/02; C07H 21/04

(52) U.S. Cl. 530/350; 536/23.7

(57) ABSTRACT

The present invention is a composition that comprises at least two hepatitis B virus surface antigens (HBsAgs), fragments thereof and/or nucleic acids encoding them, the HBsAgs differing in HBV genotype in the S region and/or pre-S1 region and the composition containing no HBV core antigen (HBcAg) or nucleic acid encoding that antigen. The present invention also includes pharmaceutical compositions, especially vaccines, comprising these compositions for the prevention and/or treatment of an HBV infection or an HBV-mediated disease. The present invention further includes a method of preparing a patient-specific medicament for the therapeutic treatment of hepatitis B.

### CROSS-REFERENCE TO RELATED APPLICATIONS

This is a divisional application of U.S. application Ser. No. 11/365,210, filed Feb. 28, 2006, which is a continuation of PCT/EP2004/009590, filed Aug. 27, 2004, which claims the benefit of German Priority Application No. 103 39 927.5, filed Aug. 29, 2003, the contents of which are hereby incorporated by reference in their entirety.

### REFERENCE TO SEQUENCE LISTING SUBMITTED ELECTRONICALLY

The official copy of the sequence listing is submitted electronically via EFS-Web as an ASCII formatted sequence listing with a file named 407133_Sequence.txt, created on Jul. 7, 2011, and having a size of 7.25 KB and is filed concurrently with the specification. The sequence listing contained in this ASCII formatted document is part of the specification and is herein incorporated by reference in its entirety.

### FIELD OF THE INVENTION

The present invention relates to compositions that comprise at least two hepatitis B virus surface antigens (HBsAgs), fragments thereof and/or nucleic acids encoding them, the HBsAgs differing in hepatitis B virus (HBV) genotype in the S region and/or pre-S1 region of HBsAg, the composition containing no HBV core antigen (HBcAg) or nucleic acid encoding that antigen; to pharmaceutical compositions, especially vaccines comprising those compositions and their use in the prevention/treatment of an HBV infection or an HBV-mediated disease. The present invention relates also to a method of preparing a patient-specific medicament for the therapeutic treatment of hepatitis; and to a kit for the diagnosis of HBV genotypes.

### BACKGROUND OF THE INVENTION

More than 250 million people worldwide are infected with the hepatitis B virus (HBV). A significant number of those infected exhibit pathological consequences, including chronic hepatic insufficiency, cirrhosis and hepatocellular carcinoma (HCC). The reason why certain people develop an acute HBV infection, while others do not, is little understood. Clinical data and analogy with other chronic viral infections have stressed the significance of a cell-mediated immune response in the control of viral infections, especially an immune response that includes cytotoxic T-lymphocytes. The induction of a cytotoxic T-cell response is a critical factor in eliminating acute HBV infection and preventing chronic HBV infection. The viral genome encodes inter alia the envelope proteins pre-S1, pre-S2 and the S-antigen (HBsAg), the polymerase and the core protein (HBcAg).

Chronic hepatitis B is progredient inflammation of the liver which can take a chronically persistent or chronically aggressive course. Chronically persistent hepatitis exhibits infiltration confined to the broadened portal areas of the liver with increasing fibrosation; clinically, signs of persistent hepatitis remain for years (up to 10 years), about 80% of the cases being HBsAg-positive. The pathogenesis is probably based on insufficiency of the cellular immune system and persistent viral infection.

The small hepatitis B surface antigen (HBsAg), a 226 amino acid protein (p24/gp27 or S-protein), is a prominent HBV antigen which is itself assembled in 20-30 nm lipoprotein particles in which 100-150 subunits are crosslinked by multiple inter- and intra-molecular disulfide bonds. The variability of the S-protein from HBV-isolates of different subtypes and genotypes is limited. The four stable, HBsAg subtypes adw, ayw, adr and ayr relate to single amino acid exchanges at positions 122 and 160 which are located adjacent to the immunodominant “a-determinant” (a hydrophilic region comprising residues 124-147). Those subtypes have not previously been assigned any biological or pathogenetic differences in HBV infection.

A vaccine obtained from the plasma of chronic HBsAg carriers was approved for the first time in the Federal Republic of Germany in 1982. Since that time, the vaccine has been produced by genetic techniques and used for the active immunisation of groups at risk. 95% of people who are seronegative prior to inoculation exhibit an immune reaction after one year. All hepatitis B vaccines used contain a high concentration of the purified HBsAg protein corresponding to the non-infectious sheath of the hepatitis B virus and are free of viral DNA or are formalin-deactivated.

A disadvantage of the prior art is that at least 5% of people that are immunised are “non-responders” who do not exhibit an immune response. Furthermore, there has been no known vaccine hitherto for the treatment of chronically persistent hepatitis.

WO 01/40279 and WO 01/38498 describe vaccines based on hepatitis B virus genotype G, but the two patent specifications make no mention of a combination of different genotypes.

Michel et al., PNAS 92 (1995), 5307-5311 and Mancini et al., PNAS 93 (1996), 12496-12501 relate to DNA vaccines based on HBsAg. The documents make no mention of the use of compositions that contain combinations of HBsAg of different HBV genotypes.

### SUMMARY OF THE INVENTION

The present invention is therefore based on the problem of providing improved means of preventing/treating an HBV infection or an HBV-mediated disease. The present invention is also based on the problem of providing a patient-specific medicament for the therapeutic treatment of hepatitis. A further objective is to provide an improved kit for the diagnosis of HBV infections.

The problem underlying the present invention is solved by the provision of a composition comprising at least two hepatitis B virus surface antigens (HBsAgs), fragments thereof and/or nucleic acids encoding them, the HBsAgs differing in hepatitis B virus (HBV) genotype in the S region and/or pre-S1 region of HBsAg, the composition containing no HBV core antigen (HBcAg) or nucleic acid encoding that antigen.

The present invention is based on the following surprising observation: transgenic mice that express constitutively the HBsAg subtype ayw in the liver are regarded as being a preclinical model for assessing the efficiency of specific immuno-therapy protocols for chronic HBV infections. Such mice produce large amounts of HBsAg, which occurs as a result of persistent antigenaemia, and are substantially tolerant with respect to HBsAg. The inventors have now immunised HBsAg-transgenic mice on the one hand with a vaccine that corresponds in its HBsAg genotype exactly to the genotype of the transgenic mouse (ayw) and, on the other hand, with a vaccine that contains an HBsAg genotype different from that of the transgenic mouse. Despite repeated immunisation of the transgenic mouse with an HBsAg antigen that corresponds to its own HBsAg, no cytotoxic T-cell response was observed. In contrast, immunisation of transgenic mice with an HBsAg genotype different from their own genotype resulted in genotype-specific and cross-reactive cytotoxic T-cell responses to HBsAg. This shows that a naturally occurring variant of HBsAg can break “tolerance” by the priming of a cross-reactive T-cell immunity. Activation of the cytotoxic T-cell immunity results in a decrease in the HBsAg ayw-antigen and, furthermore, in liver-specific signs and symptoms which correspond to acute hepatitis with effective control of the HBV. The immune response observed is especially remarkable because the amino acid sequence of the HBsAg ayw-antigen differs from the amino sequence of the HBsAg adw2-antigen only at a small number of positions. It has been ascertained in the present invention that even a small number of conservative exchanges of amino acids in a T-cell epitope may result in a change in the T-cell reaction with respect to that epitope.

The specificity and efficiency of the T-cell response to a protein antigen is regulated on various levels, especially decisive factors being: (i) the proteolytic release of the epitope (or antigenic peptide); (ii) the affinity of the antigenic peptide for the presenting glycoprotein of the major histocompatibility complex (MHC); and (iii) the negative interference of competitively developing T-cell responses to different epitopes of the same antigen. Natural variants of a protein antigen can (by individual amino acid exchanges in critical sequences within the epitope or flanking the epitope, or by creation of new epitopes) induce a specific T-cell response in the following four ways:
(i) more efficient proteolytic processing (release) of the antigenic peptide from the protein;
(ii) high-affinity binding of the antigenic peptide to the presenting MHC molecule;
(iii) elimination of immunodominant epitopes (which suppress responses to other epitopes of the same protein antigen) by an analogous progress, mentioned in (i) and/or (ii), which weakens the immunogenicity of the epitope;
(iv) new epitopes can be generated.

In the context of the present invention it is demonstrated that natural variants of HBsAg, reflected by the genotypes, have a relatively broad spectrum of specificities in the T-cell response which they stimulate.

In connection with the present invention, the term “HBV genotype” means the totality of the hepatitis B virus genome. The HBV genotype is preferably determined by total sequencing and phylogenetic analysis. At the present time 8 standard genotypes are known. Those 8 genotypes are based on a nucleotide variation of 8% with respect to one another; see Bartholomeusz, Rev. Med. Virol. 13 (2003), 1-14. Preferably the HBV genotype A has the reference nucleic acid sequence Genbank X02763 or, for the HBV genotype A_{afr}, the reference nucleic acid sequence in accordance with Genbank AF297621. For the HBV genotype Bₐ, the reference nucleic acid sequence is Genbank D00330 and for the genotype Bj the reference nucleic acid sequence is AB073858. For the HBV genotype C, the reference nucleic acid sequence is Genbank AY206389, and in respect of the genotype Cₐᵤₛ the reference nucleic acid sequence according to Genbank AB048704. For the genotype D, the reference nucleic acid sequence is Genbank X02496. The reference nucleic acid sequence for the genotype E is X75657. The reference nucleic acid sequence for the genotype F is X69798. The reference nucleic acid sequence for the genotype G is AF160501 and the reference nucleic acid sequence for the genotype H is AY090454.

In respect of the above-mentioned genotypes, there is a certain correlation between genotype and subtype as follows: genotype A correlates with subtype adw2, ayw1; genotype B correlates with adw2, ayw1; genotype C correlates with adw2, adrq+, adrq−, ayr, adr. Genotype D correlates with ayw2, ayw3, ayw4. Genotype E correlates with ayw4. Genotype F correlates with adw4q−, adw2 and ayw4; genotype G correlates with adw2 and genotype H correlates with adw4.

The determination of the HBV subtype of an infected patient can be carried out serologically with the aid of mono-specific antibodies, for example anti-d, anti-y, anti-r, anti-a(w). The determination can be effected in the form of an agar gel diffusion test or in the form of a radio immunoassay; (“HBs Antigen Subtypes”, published by: Couroucé, A. M., Holland, P. V., Muller, J. Y. and Soulier, J. P., Bibliotheca Haematologica no. 42, S. Karger, Basel, 1976).

The subtype can also be determined by sequencing the HBsAg-encoding DNA from patient serum. The amino acid sequence of the HBsAg is then derived from the determined nucleic acid sequence. The assignment of the subtype is then carried out by means of the amino acids at positions 122 and 160 as described in Magnius, L. O. and Norder, H., “Subtypes, Genotypes and molecular epidemiology of the hepatitis B virus as reflected by sequence variability of the S-gene” Intervirology 38(1-2): 24-34.

In connection with the present invention, the expression “hepatitis B virus surface antigen” (HBsAg) denotes the small HBV surface antigen or S protein (p24/gp27). HBsAg can also include the pre-S1 protein domain. Preferably, HBsAg consists of the S protein and/or the pre-S1 protein domain.

In respect of the numbering of HBsAg, the system in accordance with Kidd-Ljunggren et al., J. Gen. Virol. 83 (2002), 1267-1280, is used.

The term “fragment” includes according to the invention fragments of HBsAg. The fragment preferably comprises at least 5 amino acids and contains a T-cell epitope, preferably at least 10, especially at least 20, more especially at least 50 amino acids. In accordance with a preferred embodiment, the composition comprises at least two HBsAgs or two fragments thereof. Such a composition is especially suitable for use as a polypeptide-based vaccine. Particularly in the case where the composition comprises two fragments that are derived from HBsAgs with a different HBV genotype, the first and the second fragments have at least 10 amino acids, preferably 20 amino acids, in common, but differ from one another by at least one amino acid.

As mentioned above, the present invention is based on the recognition that even very small differences in an antigen (HBsAg) as a result of different genotypes lead to modified T-cell epitopes which differ only very slightly from one another but result in a dramatic change in T-cell reactivity. The two fragments which differ from one another by at least one amino acid can therefore readily be detected by simple sequence comparison of the known genotypes in respect of the HBsAg. Suitable fragments that differ from one another by at least one amino acid can be used in the composition according to invention. The fragments preferably contain at least one T-cell epitope, especially a human cell epitope. Methods of determining T-cell epitopes are known, for example Lauer et al., J. Virol. 76 (2002), 6104-6113.

In accordance with a preferred embodiment, the composition comprises at least two HBsAgs and/or at least two fragments thereof.

Preference is also given to compositions that comprise at least a first HBsAg or a fragment thereof and a nucleic acid encoding a second HBsAg or a fragment thereof, the first and the second HBsAgs differing in HBV genotype.

In accordance with a further preferred embodiment, the composition comprises at least two nucleic acids that encode two HBsAgs, the HBsAgs differing in HBV genotype.

The nucleic acids can also be nucleic acids that encode a fragment as defined above. The nucleic acids may be viral DNA or synthetic DNA, synthetic DNA sequences being understood as including those which contain modified internucleoside bonds. The nucleic acids can also be RNA molecules, which may be necessary for expression by means of recombinant vector systems. Furthermore, in accordance with the invention, mixed DNA/RNA molecules also come into consideration as nucleic acids.

In accordance with a preferred embodiment, the genotype is selected from the known genotypes A, B, C, D, E, F, G and H. In respect of the respective reference nucleic acid sequence, reference is made to the above definition section. The genotype is usually determined by means of an 8% nucleotide variation relative to the reference nucleic acid sequence, that is to say nucleic acids that are at least 92% identical to the reference nucleic acid sequence are also understood as a genotype in accordance with the definition. Identity of at least 95%, especially 98%, relative to the reference nucleic acid sequence is especially preferred. “Identity” relative to the reference nucleic acid sequence is here determined with the aid of known methods. Special computer programs having algorithms taking account of specific requirements are generally used.

Preferred methods of determining identity generate in the first instance the greatest agreement between the sequences being compared. Computer programs for determining identity include, but are not limited to, the GCG program package, including GAP (Deveroy, J. et al., Nucleic Acid Research 12 (1984), 387; Genetics Computer Group University of Wisconsin, Medicine (WI); and BLASTP, BLASTN and FASTA (Altschul, S., et al. J. Mol. Biol. 215 (1990), 403-410. The BLASTX program can be obtained from National Center For Biotechnology Information (NCBI) and from other sources (BLAST Handbook, Altschul S. et al., NCBI NLM NIH Bethesda N. Dak. 22894; Altschul S. et al.; above). The known Smith-Waterman algorithm can likewise be used for determining identity.

Preferred parameters for nucleic acid comparison include the following:

Needleman and Wunsch algorithm, J. Mol. Biol. 48 (1970), 443-453

Comparison matrix:

Matches=+10

Mismatches=0

Gap penalty: 50

Gap length penalty: 3

The GAP program is likewise suitable for use with the above parameters. The above parameters are the default parameters in nucleic acid sequence comparison. Further examples of algorithms, gap opening penalties, gap extension penalties and comparison matrices include those in the program handbook Wisconsin Package, Version 9, September 1997. The choice depends upon the comparison being carried out and also upon whether the comparison is being carried out between pairs of sequences, when GAP or Best Fit are used, or between a sequence and a large sequence data bank, when FASTA or BLAST are used. 92% agreement in accordance with the above algorithm represents 92% identity in connection with the present invention. The same applies to higher identities.

The composition according to the invention is preferably characterised in that the variant encodes a polymerase the activity of which corresponds substantially to the activity of the polymerase encoded by the reference nucleic acid sequence and/or the variant encodes an HBsAg the immunoreactivity of which corresponds substantially to the immunoreactivity of the HBsAg encoded by the reference nucleic acid.

The polymerase activity can here be determined in accordance with Kim et al., Biochem. Mol. Biol. Int. 1999; 47 (2), 301-308. The immunoreactivity of HBsAg can be determined by commercially available antigen ELISAs. A “substantially by the immunoreactivity of the HBsAg encoded by the reference nucleic acid” means that an antibody binds to the reference HBsAg with substantially the same affinity as to the HBsAg encoded by the variant.

In accordance with a preferred embodiment, the composition comprises at least three, preferably at least five, different HBsAgs, fragments thereof and/or nucleic acids encoding them.

Especially preferably, the composition comprises HBsAgs of all known HBV genotypes, fragments thereof and/or nucleic acids encoding them.

In accordance with a further preferred embodiment of the composition according to the invention, the nucleic acid encoding HBsAg or a fragment thereof is present in a vector under the control of a promoter suitable for expression of HBsAg in a mammal cell, preferably a human cell. If the composition comprises at least two nucleic acids encoding HBsAg or a fragment thereof, those acids can be present in the same vector (binary vector) or separately from one another on different vectors. Suitable vectors are, for example, plasmids, adenoviruses, vaccinia viruses, baculoviruses, measles viruses and retroviruses. The vector generally comprises a replication source which effects the replication of the vector in the transfected mammal cell.

Suitable promoters can be both constitutive and inducible promoters. Preferred promoters are derived from CMV and SV-40.

The compositions described above can be obtained by simply mixing the individual components and are therefore very simple to prepare. Suitable solvents and carriers depend upon the nature of the composition (polypeptide and/or nucleic acids). In principle, water-containing systems are preferred. HBsAg or fragments thereof are obtainable synthetically or by recombinant preparation. The polypeptides prepared can be purified by chromatographic methods.

Alternatively, the compositions can be obtained by co-expression of the at least two nucleic acids encoding HBsAg or fragments thereof in a recombinant expression system. The person skilled in the art will be familiar with numerous expression systems and methods; preferably yeast is used as host cell, especially preferably Hansenula acids can be present within a vector or in two vectors that are separate from one another. Suitable vectors and promoters are as described above.

In accordance with a further aspect of the present invention, pharmaceutical compositions are prepared that comprise a composition according to the invention and a pharmaceutically acceptable carrier. Pharmaceutically acceptable carriers are known to the person skilled in the art. Examples are: aluminium salts, calcium phosphate, lyophilisates of HBsAg with or without addition of polysaccharide, oil-in-water emulsions, poly-lactide-co-glycolate. Where such carriers do not themselves have an adjuvant action, they can be admixed with further adjuvants, such as, for example, lipid A mimetics, immunostimulatory nucleotides or bacterial toxins.

The pharmaceutical composition according to the invention is especially a vaccine. According to the invention, the pharmaceutical composition, especially the vaccine, is suitable for the therapeutic treatment of an HBV infection or an HBV-mediated disease. The pharmaceutical composition, especially the vaccine, is also suitable for the prophylactic treatment of an HBV infection or an HBV-mediated disease.

The HBV infection is especially a chronically persistent hepatitis B infection. An HBV-mediated disease can be an acute chronic hepatitis B infection. Further HBV-mediated diseases are cirrhosis of the liver and primary liver cell carcinoma. The vaccine is suitable for administration to clinically inapparent HBV carriers, that is to say carriers who are not yet suffering from disease in the true sense, but have a high risk of developing an HBV-mediated disease in the future.

The pharmaceutical composition can be administered intramuscularly, subcutaneously, intradermally, intravenously, mucosally or orally, but such administration is merely indicated as being preferred and there is no limitation thereto. The pharmaceutical composition comprises the at least two HBsAgs or fragments thereof in a dosage range of from 0.1 to 1000 μg/HBsAg or fragment thereof, preferably from 2.5 to 40 μg/HBsAg or fragment thereof.

When the pharmaceutical composition comprises nucleic acids encoding HBsAg or fragments thereof, they are present in a dosage range of from 10 to 1000 μg/nucleic acid encoding HBsAg or fragments thereof.

A further aspect of the present invention provides a method of preparing a medicament for the therapeutic treatment of hepatitis B which comprises the following steps:

a) determination of the HBV genotype with which the patient is infected; and

b) provision of a medicament comprising at least one HBsAg of an HBV genotype, a fragment of the HBsAg or a nucleic acid encoding HBsAg or a fragment thereof, the HBV genotype differing from the HBV genotype of the patient determined according to a).

As mentioned above, an important recognition of the present invention is that in a preclinical model of chronically persistent hepatitis a treatment effect has been obtained by treating the transgenic animal with an HBsAg originating from an HBV genotype that differs from the genotype of the transgenic animal.

The genotype can be determined by the following methods: sequencing of the total HBV genome or at least the portion coding for the HBsAg and phylogenetic analysis, restriction fragment length polymorphism (RFLP), multiplex-PCR.

The provision of the medicament is carried out in a manner known per se by formulation of at least one HBsAg, a fragment thereof or a nucleic acid encoding HBsAg of a fragment thereof.

In accordance with a further aspect, the present invention provides a kit for diagnosis of the genotype of an HBV infection. The kit comprises at least two HBsAg-specific binders, characterised in that the two HBsAg-specific binders are specific to different HBV genotypes. The at least two HBsAg-specific binders can be HBsAg genotype-specific primers and/or specific antibodies. The primers can have a length of 10-30 nucleotides and are complementary to the known HBsAg-sequences of the respective genotype. The antibodies are antibodies that can be obtained, for example, by immunisation of experimental animals, such as, for example, mice having the respective HBsAg corresponding to the desired HBsAg genotype, preparation of hybridomas in a manner known per se and screening for subtype-specific monoclonal antibodies.

### BRIEF DESCRIPTION OF THE DRAWINGS

FIG. 1: HBsAg variants. (A) The amino acid sequence of the small hepatitis B surface antigen (HBsAg) ayw (1) corresponding to genotype D and adw2 (2) corresponding to genotype A are shown. (B) HBsAg ayw- and adw2-derived, K^{b}-restricted epitope sequences. The epitope 1 (S₂₀₈₋₂₁₅) was presented only by the cells that process exogenous HBsAg, whereas epitope 2 (S₁₉₀₋₁₉₇) was presented only by the cells that process endogenous HBsAg.

FIG. 2: Transfer of epitope-1- or epitope-2-specific cytotoxic T-cell lines (CTLL) into HBs-transgenic (HBs-tg) hosts lead cytotoxic T-cell lines HBs-transgenic to transient liver damage. The spleen cells were removed from pCl/S_{ayw}, DNA-immunised B6 mice and restimulated in vitro with syngenic RBL5-cells, the RBL5-cells being pulsed with K^{b}/S₂₀₈₋₂₁₅-binding peptide 1 (ILSPFLPL) or K^{b}/S₁₉₀₋₁₉₇-binding peptide 2 (VWLSVIWM), or stimulated with ConA. 5×10⁶ CD8⁺ CTLL/mouse were injected intravenously (i.v.) into HBs-tg mice and the average serum alanine transminase (ALT) level was determined.

FIG. 3: Ex vivo demonstration of HBsAg-specific CD8⁺ T-cells in the liver and spleen of immunised mice. C57BL/6 mice were immunised intramuscularly by a single injection of 100 μg of pCl/S_{ayw} DNA. Specific CD8⁺ T-cells were demonstrated 12 days after immunisation. Isolated liver-mononuclear cells (MNC) and spleen cells were restimulated in vitro over a period of four hours (in the presence of Brefeldin A) with the K^{b}/S₂₀₈₋₂₁₅-binding peptide 1 (ILSPFLPL) or the K^{b}/S₁₉₀₋₁₉₇-binding peptide 2 (VWLSVIWM). The average frequency of CD8⁺ IFNγ⁺ T-cells/10⁵ CD8⁺ T-cells±standard deviation of 4-6 mice (from two experiments that are independent of one another) per group is shown.

FIG. 4: HBsAg-specific CD⁸ T-cell responses to the epitope 1 in HBs-tg mice. HBs-tg mice which express HBsAg_{ayw} in the liver were immunised intramuscularly three times (at four-week intervals) with DNA vaccines that encode HBsAg subtype ayw (pCl/S_{ayw}) or adw2 (pCl/S_{adw2}) or with the negative control vector pCl (vector without insert). The spleen cells were removed from the immunised mice 12 days after the last immunisation and were restimulated over a period of four hours in vitro (in the presence of Brefeldin A) with RBL5 cells, the RBL5 cells being restimulated with HBsAg particles of the ayw (RBL5/S^{P}_{ayw}) or adw2 (RBL5/S^{P}_{adw2}) subtype, or with the K^{b}/S₂₀₈₋₂₁₅-binding peptide 1 of HBsAg_{ayw} (ILSPFLPL) or HBsAg_{adw2} (IVSPFIPL). The average number of spleen IFNγ⁺ CD8⁺ T-cells/10⁵ CD8⁺ T-cells±standard deviation of 4 to 6 mice (from two experiments that are independent of one another) per group is shown.

FIG. 5: HBsAg-specific CD⁸ T-cell responses to epitope 2 in HBs-tg mice. The spleen cells were removed from mice that had been immunised as described in respect of the legend of FIG. 4, and were restimulated in vitro with syngenic RBL5/S_{ayw} or RBL5/S_{adw2} transfectants, or with the K^{b}/S₁₉₀₋₁₉₇ epitope 2 of HBsAg_{ayw} (VWLSVIWM) or HBsAg_{adw2} (VWLSAIWM). The average numbers of spleen IFNγ⁺ CD8⁺ T-cells/10⁵ CD8⁺ T-cells±standard deviation of 4 mice per group is shown.

FIG. 6: S₂₀₈₋₂₁₅-specific CD8⁺ T-cells were demonstrated in the liver of immunised HBs-tg mice. Transgenic HBs-tg mice were immunised three times (at 4-week intervals) with a DNA vaccine encoding HBsAg_{adw2} (pCl/S_{adw2}). Liver and spleen cells were removed from immunised mice 12 days after the last injection and restimulated in vitro with the K^{b}/S₂₀₈₋₂₁₅-binding peptide ILSPFLPL. The average number of spleen IFNγ⁺ CD8⁺ T-cells/10⁵ CD8⁺ T-cells±standard deviation of 4 mice per group is shown.

FIG. 7: Liver histopathology of HBs-tg mice that have been immunised with the pCl/S_{adw2} DNA vaccine. Non-pathological liver histology was observed in B6 mice (A, B). HBs-tg mice (C, D) exhibited moderate cell enlargement and the cytoplasm exhibits a ground glass appearance (D). The nuclei of the liver cells appeared moderately polymorphic. Periportal infiltrations are rare. Repeated immunisation with pCl/S_{adw2} DNA induces severe histomorphological changes in the liver (E-I) which are consistent with acute viral hepatitis. Inflammatory infiltrations include Kupfer cells, lymphocytes and a small number of polymorphonuclear granulocytes which are located in the lobular parenchyma (F) and in the periportal areas (G). The hepatocytes appear hydropic and often have pyknotic nuclei, which is a sign of an early stage of apoptosis (F, arrows). Acidophilic bodies (H, arrows), that is to say apoptotic liver cells, are common and often surrounded by focal inflammatory infiltrations. Many liver cells exhibit marked vacuolisation (I, arrows). H & E staining of formalin-fixed, paraffin-embedded tissue. Original magnifications: ×10 in A, C and E; ×40 in B, D and F; ×63 in G-I.

FIG. 8: Induction of HBsAg-specific serum antibody responses in HBs-tg mice. B6 mice and transgenic HBs-tg mice were immunised intramuscularly with DNA vaccines that encode HBsAg_{adw2} (pCl/S_{adw2}) or HBsAg_{ayw} (pCl/S_{ayw}) and after three weeks are boosted with the same vaccines. Four weeks after the last injection, serum samples were tested for HBsAg antigen (A) or HBsAg-specific antibodies (B). The average antibody titres (mIU/ml) and serum HBsAg levels (ng/ml)±standard deviations of 4-6 mice/group are shown.

FIG. 9:

HBsAg-specific CD8⁺ T-cell responses to epitope 1 (S₂₀₈₋₂₁₅) and to epitope 2 (S₁₉₀₋₁₉₇) in normal B6 and HBs_{ayw}-tg mice.

The animals were each immunised three times (at 21-day intervals) intra-muscularly with HBsAg protein particles (S^{P}) of the subtype ayw or adw2. The protein vaccines were each admixed with CpG-oligonucleotides (ODN) or RC-529 as adjuvant. PBS was used as negative control. The spleen was removed from the animals 12 days after the last immunisation and the isolated spleen cells were then restimulated over a period of four hours in vitro (in the presence of Brefeldin A) with RBL5 cells which had been pulsed beforehand with HBsAg-specific peptides. For that purpose, in each case the K^{b}/S₂₀₈₋₂₁₅-binding peptide 1 of HBsAg_{ayw} (ILSPFLPL) or HBsAg_{adw2} (IVSPFIPL) or the K^{b}/S₁₉₀₋₁₉₇-binding peptide 2 of HBsAg_{ayw} (VWLSVIWM) or HBsAg_{adw2} (VWLSAIWM) was used. The number of spleen IFNγ⁺ CD8⁺ T-cells/10⁵ CD8⁺ T-cells±standard deviation of 4-6 mice (from two experiments that are independent of one another) per group is shown.

FIG. 10:

HBsAg-specific CD8⁺ T-cell responses to the epitope 1 (S₂₀₈₋₂₁₅) in HBs_{ayw}-tg mice.
A. HBs-tg mice which express HBsAg_{ayw} in the liver were immunised intramuscularly three times (at four-week intervals) with DNA vaccines that code solely for HBsAg subtype ayw (pCl/S_{ayw}) or for the three subtypes ayw (pCl/S_{ayw}), adw₂ (pCl/s_{adw2}) and adr (pCl/S_{adr}), or with the negative control vector pCl (vector without insert). The spleen was removed from the animals 12 days after the last immunisation. The isolated spleen cells were restimulated over a period of 4 hours in vitro (in the presence of Brefeldin A) with RBL5 cells that had been pulsed beforehand with the K^{b}/S₂₀₈₋₂₁₅-binding peptide 1 of HBsAg_{ayw} (ILSPFLPL) or HBsAg_{adw2} (IVSPFIPL). The number of spleen IFNγ⁺ CD8⁺ T-cells/10⁵ CD8⁺ T-cells±standard deviation of 4-6 mice (from two experiments that are independent of one another) per group is shown.
B. A. HBs_{ayw}-tg mice were immunised intramuscularly three times (at 21-day intervals) intramuscularly with HBsAg protein particles (S^{P}) of subtype ayw or a mixture of HBsAg protein particles of subtypes ayw, adw2 and adr. The protein vaccines were each admixed with CpG-oligonucleotides (ODN) or RC-529 (shown only for subtype mixture) as adjuvant. PBS was used as negative control. The spleen was removed from the animals 12 days after the last immunisation. The isolated spleen cells were restimulated over a period of 4 hours in vitro (in the presence of Brefeldin A) with RBL5 cells that has been pulsed beforehand with the K^{b}/S₂₀₈₋₂₁₅-binding peptide 1 of HBsAg_{ayw} (ILSPFLPL) or HBsAg_{adw2} (IVSPFIPL). The number of spleen IFNγ⁺ CD8⁺ T-cells/10⁵ CD8⁺ T-cells±standard deviation of 4-6 mice (from two experiments that are independent of one another) per group is shown.

FIG. 11:

Induction of HBsAg-specific serum antibody responses in HBs-tg mice.
B6 mice and transgenic HBs-tg mice were immunised intramuscularly with HBsAg protein particle vaccines (S^{P}) of subtype ayw or of subtype adw2 or with a mixture of the subtypes ayw, adw2 and adr and after three weeks boosted with the same vaccine. The protein vaccines contained as additive CpG-oligonucleotide (ODN) as adjuvant. Four weeks after the booster injection, serum samples were tested for HBsAg (A) and HBsAg-specific antibodies (B). The average antibody titres (mIU/ml) and the serum HBsAg level (ng/ml)±standard deviations of 4-6 mice/group are shown.

### DETAILED DESCRIPTION OF THE PREFERRED EMBODIMENTS

The invention will be described in greater detail below with reference to Examples. The Examples are not intended to limit the invention, however.

### EXAMPLES

### Material and Methods

### General

The HBV subtype adw2 under investigation corresponds to genotype A. The HBV subtype ayw corresponds to genotype D. The HBV subtype adr corresponds to genotype C.

### Mice

C57BL/6JBom (B6) mice (H-2^{b}) were kept under standard-pathogen-free conditions.

C57BL/6J-TgN(Alb1b1HBV)44Bri transgenic (HBs-tg) mice, HBsAg_{ayw}, (encoded by the HBV sequence having deposition number V01460 J02203) were obtained from The Jackson Laboratory (Bar Harbour, Me.). Male and female mice 8-16 weeks of age were used.

### Cells, Recombinant HBsAg Particles and Antigenic HBsAg Peptides

The H-2^{b} cell line RBL5 used is described in [10]. Stable RBL5 transfectants that expressed similar amounts of HBsAg_{ayw} and HBsAg_{adw2} were prepared (data not shown). Recombinant HBsAg particles of subtypes ayw, adw₂ and adr are obtainable from Rhein Biotech GmbH (Düsseldorf, Germany). The HBsAg particles prepared in the Hansenula polymorpha host strain RB10 were purified as described [3]. The synthetic K^{b}-binding S₂₀₈₋₂₁₅ ILSPFLPL (ayw) or IVSPFIPL (subtype adw2) peptides and the K^{b}-binding S₁₉₀₋₁₉₇ VWLSVIWM (ayw) or VWLSAIWM (adw2) peptides were obtained from Jerini BioTools (Berlin, Germany). The peptides were dissolved in a DMSO solution in a concentration of 10 mg/ml and were diluted with culture medium before use.

### Plasmids and DNA Immunisation

HBsAg_{ayw}, HBsAg_{adw2} and HBsAg_{adr} were cloned into the pCl (Promega) and BMGneo vectors as described [4; 5]. As DNA vaccines, the plasmids pCl/S_{ayw}, pCl/S_{adw2}, pCl/S_{adr} were used which expressed HBsAg_{ayw}, HBsAg_{adw2} and HBsAg_{adr} equally well. This was shown by immunoprecipitation of HBsAg from cells that had been transiently transfected with the DNA of those plasmids (data not shown). Differences in the immunogenicity of the HBsAg epitopes therefore cannot be clarified on the basis of different amounts of HBsAg expression by the DNA vaccine or the transfectants. For intramuscular nucleic acid immunisation, 50 μl of PBS (phosphate-buffered saline) containing 1 μg/l of plasmid DNA were injected into each tibialis anterior muscle as described [4]. Immunisation with mixtures of HBsAg subtypes was effected by injection of 50 μl of PBS containing in each case 1 μg/μl pCl/S_{ayw}, 1 μg/μl pCl/S_{adw2} and 1 μg/μl pCl/S_{adr}.

### Immunisation with HBsAg Protein Particles

5 μg of HBsAg protein particles were injected subcutaneously together with 30 μg of CpG oligonucleotide (ODN1826, MWG Biotech, Ebersberg, Germany) or 8 μg of RC-529 (Corixa Corp. Seattle, Wash., USA) in 100 μl of PBS (phosphate-buffered saline) per mouse. For immunisation with a mixture of HBsAg subtypes, in each case 5 μg of HBsAg_{ayw}, 5 μg of HBsAg_{adw2} and 5 μg of HBsAg_{adr} protein particles together with 30 μg of CpG oligonucleotide adjuvant or 8 μg of RC-529 in 100 μl of PBS were injected subcutaneously.

### Determination of Specific Spleen and Liver CD8⁺ T-Cell Frequencies

Spleen cell suspensions [1] and the preparation of hepatic NPC (non-parenchymal) cells has been described [6; 7]. The spleen cells and the liver NPC (1×10⁶1 ml) were incubated over a period of 1 hour in RPMI-1640 medium with 5 μg/μl of HBsAg-derived peptides or HBsAg-expressing transfectants (10⁶/ml) or HBsAg-particle-pulsed cells. 5 μg/μl of Brefeldin A (BFA) (catalogue No. 15870; Sigma) were then added and the cultures were incubated for a further 4 hours. The cells were harvested and their surface stained with anti-CD8 mAb, fixed and permeabilised and staining for cytoplasmic IFNγ was carried out. The frequencies of CD8⁺ IFNγ⁺ CTL were determined by FACS analysis. The average value for CD8⁺ IFNγ⁺ T-cells/10⁵ spleen or liver T-cells is shown.

### Transfer of Specific CD8⁺ T-Cell Lines

CD8⁺ T-cell lines were obtained from the spleen of B6 mice which were immunised with the pCl/S_{ayw} DNA vaccine. The spleen cells were restimulated in vitro with syngenic RBL5 cells which were pulsed with the K^{b}/S₂₀₈₋₂₁₅-binding peptide 1 (ILSPFLPL) or the K^{b}/S₁₉₀₋₁₉₇-binding peptide 2 (VWLSVIWM). In lines that were expanded in vitro over a period of about 2 weeks, more than 80% of the CD8⁺ T-cells had the expected epitope specificity, as is revealed by the specific IFNγ-expression tests. The cells were washed, and 5×10⁶ cells of those lines were injected intravenously. Control cells were non-specific CD8⁺T blasts that were isolated from 3 days ConA-stimulated cultures.

Determination of Transaminases, HBsAg and Anti-HBsAg Antibodies in Serum

Serum antibodies were repeatedly obtained from individual, immunised or control mice by removal of blood from the tail vein at certain time points after injection. The serum alanine aminotransferase (ALT) activity was carried out in the blood using the Reflotron® tests (catalogue No. 745138; Roche Diagnostics GmbH). The HBsAg concentration in the serum of the transgenic mice was determined by the commercial ELISA AUSZYME II (ABBOTT Laboratories, Wiesbaden, Germany) test. Antibodies to HBsAg were demonstrated in mouse sera using the commercial IMxAUSAB Tests (catalogue No. 7A39-20; Abbott, Wiesbaden, Germany).

Antibody levels were qualified with the aid of 6 standard sera. The tested sera were diluted so that the measured OD values lay between the standard serum one and six. The values shown herein were determined by multiplication of the serum dilution by the measured antibody level (mIU/ml). The serum titres given correspond to the mean of 4 individual mice±standard deviation.

### Histology

Thin liver tissue sections (<3 mm) were fixed in 4% formalin (pH 7.0) over a period of 24 hours and embedded in paraffin. 2 μm thick paraffin sections were stained with haematoxylin-eosin (H&E).

### Binding of HBsAg Peptides to K^{b}

Affinity-purified MHC class I molecules K^{b} were incubated over a period of 48 hours at 18° C. with increasing concentrations of test peptide and a defined concentration (about 2 nM) of radioactively labelled VSV NP 52-59 indicator peptide in the presence of 3 μM human β2 m as described [8, 9]. The binding of the peptides to MHC class I molecules was then determined by Sephadex G50 column gel filtration [8]. The radioactively labelled VSV NP 52-59 peptide was located in the exclusion volume (MHC-bound peptide) and inclusion volume (free peptide). This was determined by gamma-radiospectrometry and the proportion of the test peptide that had bound to the MHC molecule relative to the total amount of test peptide was determined. The concentration of the test peptide required to obtain 50% inhibition of the binding of the indicator peptide (IC50 value) was determined. The lower the IC50 value, the better the binding of the test peptide. In order to prevent depletion of ligand, in all binding experiments a MHC volume was used that was sufficient to obtain not more than 15-25% binding. Under those conditions, the 1050 value is an approximation to the dissociation constant (K_{d}). All binding experiments were carried out as inhibition experiments.

### Example 1

Adoptive transfer of K^{b}-restricted CD8⁺ T-cell lines that are specific to epitope 1 or epitope 2 induce liver damage in HBs-tg B6 mice

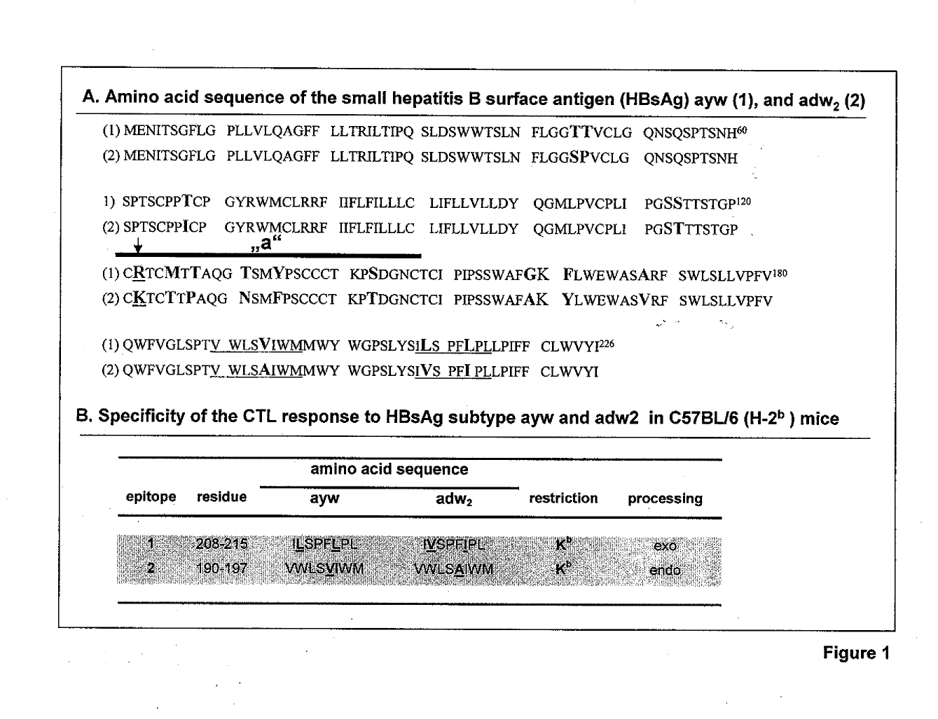

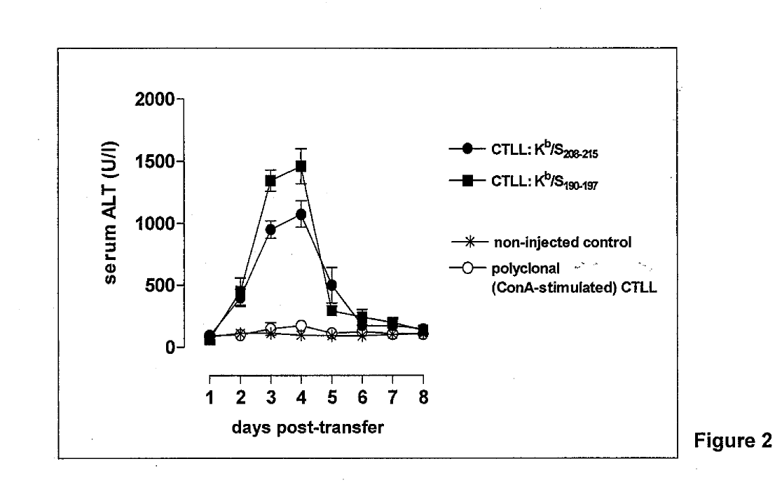

Short-term CD8⁺ T-cell lines were produced that are specific to epitope 1 or epitope 2 (FIG. 1B) of HBsAg from the spleen of B6 mice and that were immunised with pCl/S_{ayw}, plasmid DNA. Within those lines, >95% of the cells were CD8⁺, and the specific IFNγ expression was induced in >80% of those CD8⁺ T-cells. The adoptive transfer of 5×10⁶ cells of those lines into congenic B6 hosts that expressed HBsAg_{ayw}, in the liver from a transgene induced acute liver damage, as was revealed by a short, but large rise in serum transaminase (FIG. 2). The serum transaminase level normalized 5-6 days after the transfer, at which time no transferred CD8⁺ T-cells were detectable in the host. Transfer of the same number of polyclonal (mitogen-activated) CD8⁺ T blasts did not exhibit liver damage. It was therefore ascertained that (i) specific CD8⁺ T-cells effectively induce liver damage in HBs-tg mice (as described in [2]); (ii) the HBsAg epitopes, which were produced by processing of endogenous or exogeneous HBsAg, are presented in the transgene-expressing liver; and (iii) adoptively transferred CD8⁺ T-cells are rapidly removed from the transgenic host. Transferred CD8⁺ T-cells having different specificities of HBsAg therefore have access to the liver and can be activated in situ, but cannot be absorbed stably.

### Example 2

### K^{b}-restricted CTL that Recognise the HBsAg Epitopes 1 and 2 were Observed in the Spleen and Liver

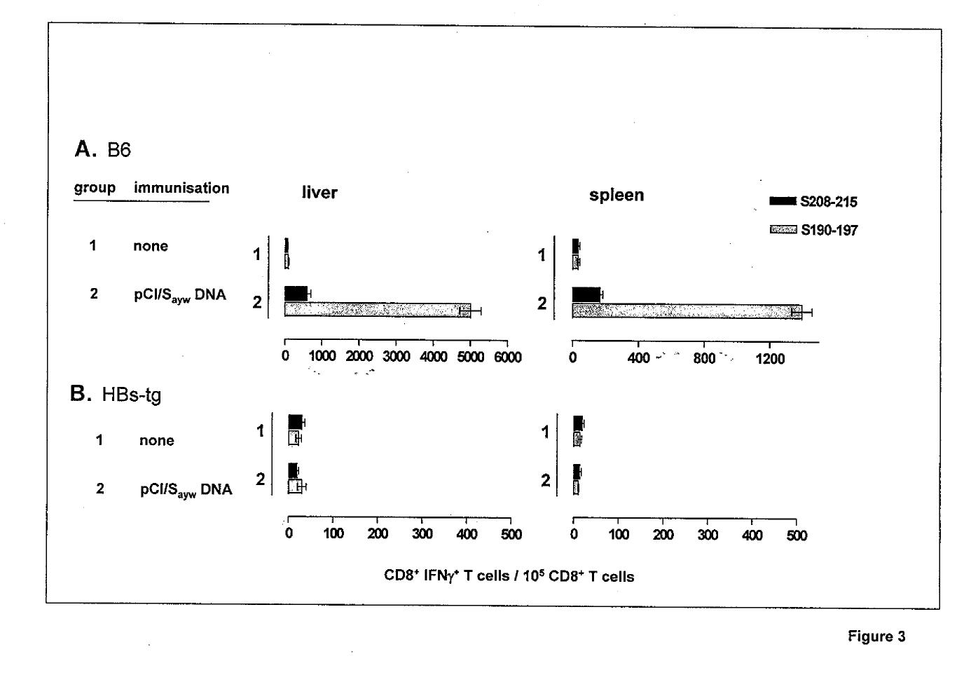

An investigation was carried out into whether vaccine-primed HBsAg-specific CD8⁺ T-cells have access to the liver in normal or transgenic HBsAg-expressing (HBs-tg) B6 mice (FIG. 3). Spleen cells and non-parenchymal liver cells (NPC) were isolated from B6 mice that had been immunised 12-15 days beforehand with the pCl/S_{ayw} vaccine. CD8⁺ T-cells that were specific to epitope 1 or epitope 2 were found in spleen and liver CD8⁺ T-cell populations from normal B6 mice (FIG. 3A). Although the frequency of HBsAg-specific CD8⁺ T-cells within the liver CD8⁺ T-cell populations was high, their absolute numbers were smaller than in the spleen (data not shown). In contrast, no CD8⁺ T-cell reactivity was demonstrable in HBsAg_{ayw} tg B6 mice that had been immunised with the DNA vaccine encoding HBsAg_{ayw} (FIG. 3B). Neither three booster injections (at three-week intervals) with the DNA vaccine nor repeated immunisations with HBsAg antigen particles and oligonucleotide adjuvant brought about HBsAg-specific CD8⁺ T-cell immunity in HBs-tg mice (data not shown). Accordingly, inoculation protocols using the same HBsAg variant to which the mouse is tolerant do not prime effective anti-viral CD8⁺ T-cell immunity.

### Example 3

### K^{b}-Restricted T-Cell Responses to the Epitopes of HBsAg_{ayw}, and HBsAg_{adw2} Variants

The HBsAg_{ayw} and HBsAg_{adw2} proteins from the HBV isolates, which proteins have 226 amino acid residues, differ in 16 amino acid residues (their amino acids accordingly being 93% identical). The sequence of the HBsAg_{ayw} protein that was used is identical to the sequence of the transgene-encoded HBsAg_{ayw} expressed by the HBs-tg B6 mice. The sequences of the K^{b}-binding epitopes 1 and 2 of HBsAg_{ayw} and HBsAg_{adw2} that were selected differ by, respectively, 1 and 2 amino acid residues within the epitope, but have identical flanking sequences (FIGS. 1A, B). The S₂₀₈₋₂₁₅-epitope 1 of HBsAg_{ayw} and HBsAg_{adw2} differ in two positions: in adw2, a valine (V) residue is replaced by a leucine (L) at position 2, and an isoleucine (I) is replaced by a leucine (L) residue at position 6 (FIG. 1B). The binding affinity of epitope 1 of K^{b} was rather low; the HBsAg_{adw2} variant of epitope 1 exhibited higher binding affinity for K^{b} than the HBsAg_{ayw} variant of the epitope (Table 1). In contrast, the binding affinity of epitope 2 for K^{b} was high (Table 1).

TABLE 1
Bindung affinity of immunogenic HBsAg epitopes for K^{b}

| | HBsAg | Peptide | K^{b}-binding |
|---|---|---|---|
| Epitope | Variant | sequence | (nM) |
| 1 | ayw | ILSPFLPL | 3400 |
| 1 | adw2 | IVSPFIPL | 773 |
| 2 | ayw | VWLSVIWM | 54 |

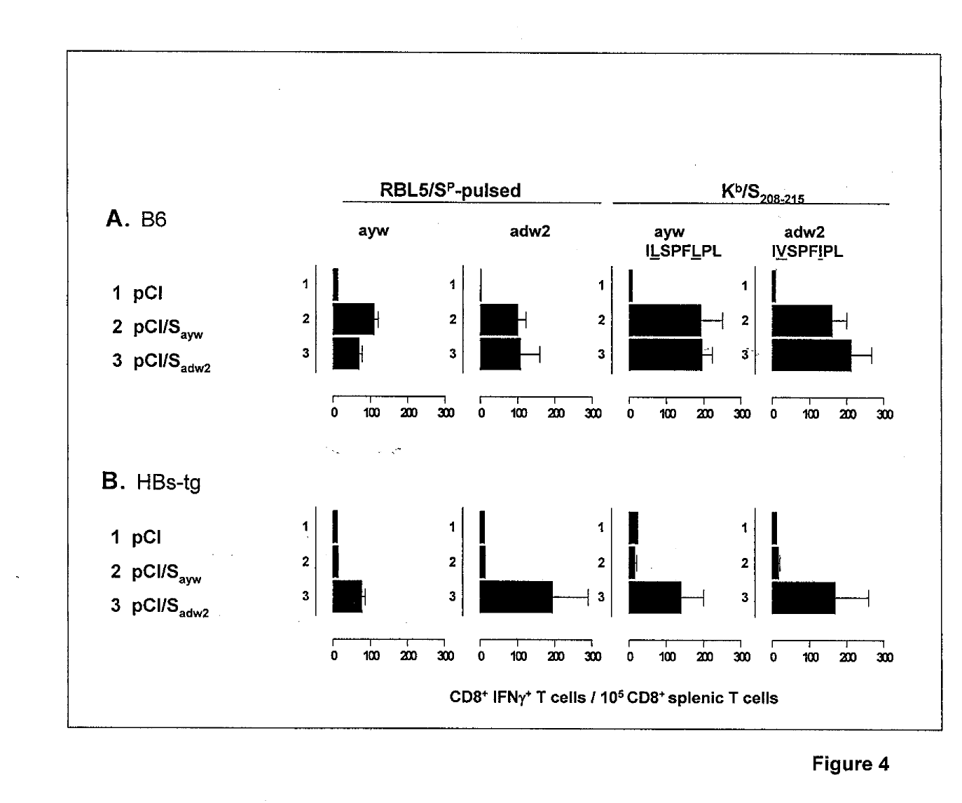

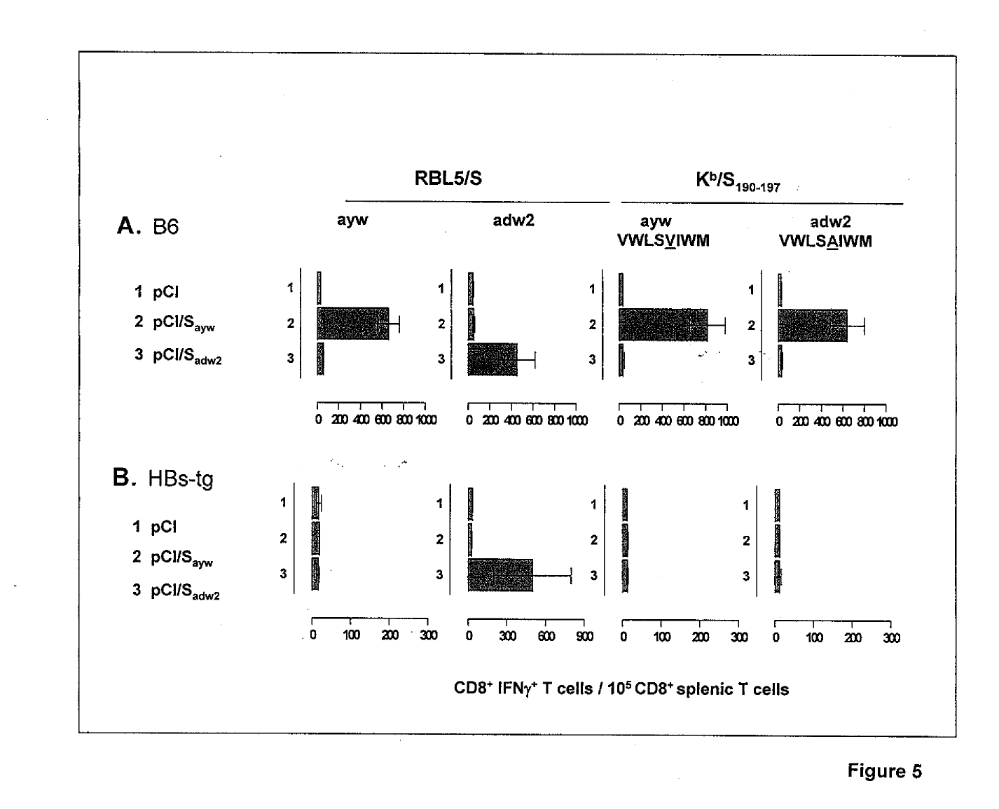

B6 mice immunised with the pCl/S_{ayw} or pCl/S_{adw2} DNA vaccine exhibited a CD8⁺ T-cell response with respect to the K^{b}-binding epitope 1 that was observed after 5 hours' ex vivo restimulation of primed spleen CD8⁺ T-cells which had been pulsed with either HBsAg_{ayw} or HBsAg_{adw2} particles or antigen peptide S₂₀₈₋₂₁₅ of HBsAg_{ayw} or HBsAg_{adw2} (FIG. 4A), group 2, 3). The ayw and adw2 variants of epitope 1 were cross-reactive, because (i) epitope-1-specific CTL were primed by pCl/S_{ayw} or pCl/S_{adw2}; and (ii) cells that had been pulsed with HBsAg_{ayw} or HBsAg_{adw2} particles or had been pulsed with peptide ILSPFLPL (ayw) or peptide IVSPFIPL (adw2) present epitope 1 to primed CD8⁺ T-cells. Accordingly, the two substitutions within the 8-mer epitope 1 did not inhibit the effective processing, K^{b}-binding or presentation of the epitope.

CD8⁺ T-cells that had been primed with the pCl/S_{ayw} DNA vaccine recognised epitope 2 (S₁₉₀₋₁₉₇) of HBsAg_{ayw} or HBsAg_{adw2} (FIG. 5A; group 2). This was demonstrated ex vivo after 5 hours' restimulation using peptide-pulsed cells or transfectants that expressed HBsAg_{ayw}. Primed CD8⁺ T-cells did not recognise transfectants that expressed the endogenous HBsAg_{adw2}. Immunisation with the pCl/S_{adw2} DNA vaccine did not prime epitope-2-specific T-cells (FIG. 5A, group 3). CD8⁺ T-cells that had been primed with pCl/S_{adw2} (but not with pCl/S_{ayw}) DNA vaccine recognised a adw2-specific epitope of unknown epitope/restriction specificity which was presented by the transfectants; this was not investigated further (FIG. 5, group 3). Replacement of the amino acid at position 5 (exchange of the hydrophobic amino acid valine V for the hydrophobic amino acid alanine A) therefore inhibits the production of epitope 2, but not its presentation by the K^{b} molecule ([1].

### Example 4

### Cross-Reactive K^{b}-Restricted CD8⁺ T-Cell Responses to HBsAg Epitope 1 are Primed in HBs-tg B6 Mice

HBs-tg B6 mice express HBsAg_{ayw} from a transgene in the liver. HBs-tg mice were immunised with HBsAg_{ayw} (pCl/S_{ayw}) or HBsAg_{adw2} (pCl/S_{adw2}) (FIGS. 4, 5B). No CD8⁺ T-cell response was obtained by repeated immunisation of HBs-tg B6 mice with the pCl/S_{ayw} DNA vaccine (FIGS. 4, 5B, group 2). In contrast, immunisation of HBs-tg B6 mice with the pCl/S_{adw2} DNA vaccine produced a CD8⁺ T-cell response to HBsAg (FIG. 4B, group 3). This cross-reactive CD8⁺ T-cell response recognised cells that had been pulsed with HBsAg_{ayw} or HBsAg_{adw2} particles or with the ayw or adw2 variant of epitope 1 in peptide form (FIG. 4B, group 3). Those CD8⁺ T-cells did not recognise the RBL5/S_{ayw} transfectants or the K^{b}-binding epitope 2 S₁₉₀₋₁₉₇ (FIG. 5B, group 3). The CD8⁺ T-cells exhibited a subtype-specific reactivity towards an undetermined determinant which was presented by RBL5/S_{adw2} but not by the RBL5/S_{ayw} transfectants (FIG. 5B, group 3). This shows that a natural variant of HBsAg is able to “break tolerance” by the priming of a cross-reactive T-cell immunity.

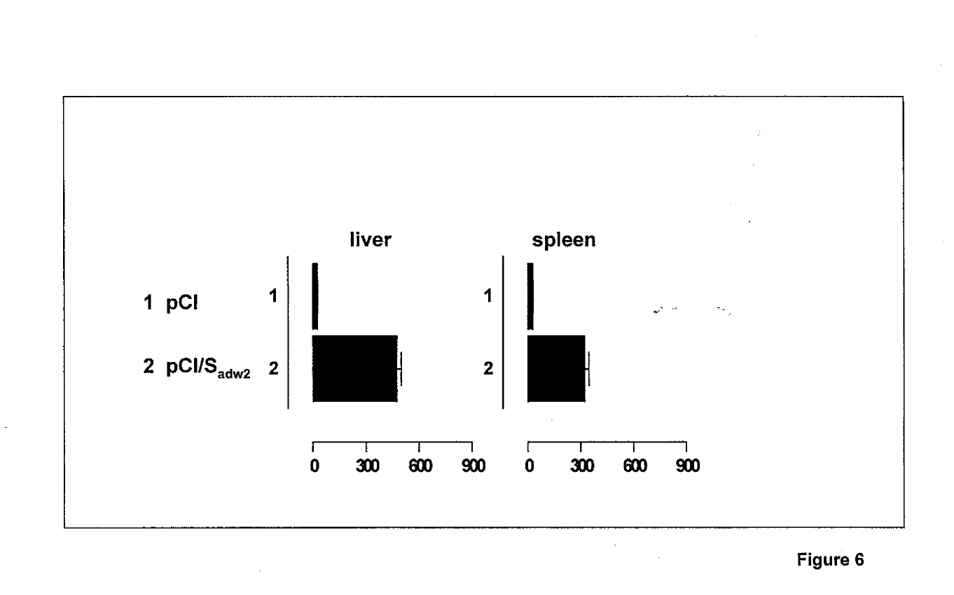

An investigation was carried out into whether specific CD8⁺ T-cell populations can be demonstrated in the antigen-producing liver in the transgenic mice which were immunised with pCl/S_{adw2}. In the spleen and in liver NMC from HBs-tg B6 mice that had been immunised with pCl/S_{adw2}, specific CD8⁺ T-cell reactivity can be demonstrated over periods of months (FIG. 6). In contrast to the adoptively transferred CD8⁺ T-cells (FIG. 2), vaccine-primed anti-HBV-specific CD8⁺ T-cells therefore have access and exhibit stable absorption into the antigen-bearing target organ over a period of more than 3 months.

### Example 5

### Histopathology of the Liver of Immunised HBs-tg Mice that Exhibit a Specific CD8⁺ T-Cell Reactivity Towards the HBsAg Epitope 1

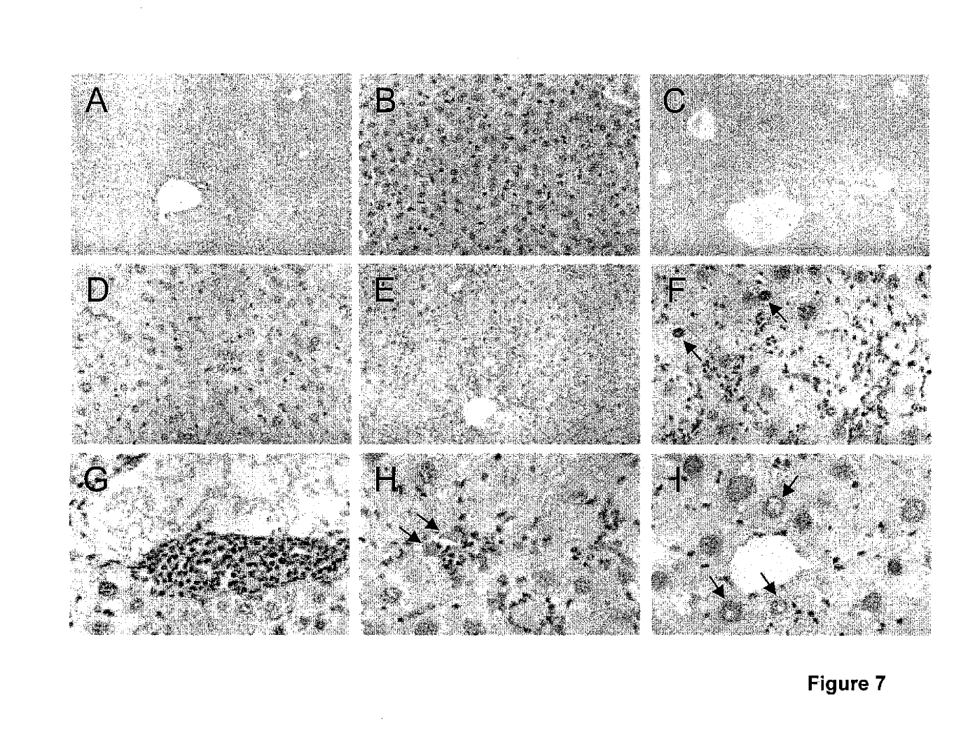

HBsAg-specific CD8⁺ T-cells induced an inflammatory response in the HBsAg-producing liver. Untreated B6 mice exhibited a normal liver histology (FIGS. 7A, B). Hepatocytes from HBs-tg B6 mice were enlarged and exhibited a fine granular, pale eosinophilic cytoplasm, which is characteristic of “ground glass liver cells” which is also observed in the case of human HBV infection (FIGS. 7C, D). No inflammatory infiltrations were observed.

HBs-tg mice that had been immunised with pCl/S_{adw2} (but not with pCl/S_{ayw}) DNA vaccine exhibited a severe liver histopathology (FIG. 7E). Inflammatory infiltrates that were found in the parenchymal (FIG. 7F) and periportal (FIG. 7G) areas consisted chiefly of mononuclear cells (FIG. 7F). Numerous small, lymphoid cells were distributed in the parenchymal and periportal areas. Localised groups of inflammatory cells surrounded the apoptotic hepatocytes (FIG. 7H). The enlargement and hydropic swelling of hepatocytes was greater in immunised HBs-tg mice than in untreated HBs-tg mice. Some medium to small nuclei exhibited a condensed chromatin and a perinuclear halo (FIG. 7F arrows), which points to an early stage of apoptosis. Furthermore, numerous Councilman's bodies, representing apoptotic liver cells, were observed (FIG. 7H, arrows). Some hepatocytes exhibited nuclear vacuolisation (FIG. 7, arrows). Significant cholestasis was not demonstrable.

### Example 6

### Priming of HBsAg-Specific Cd8⁺ T-Cells in HBs-Tg Mice Correlates with a Reduction in Antigenaemia

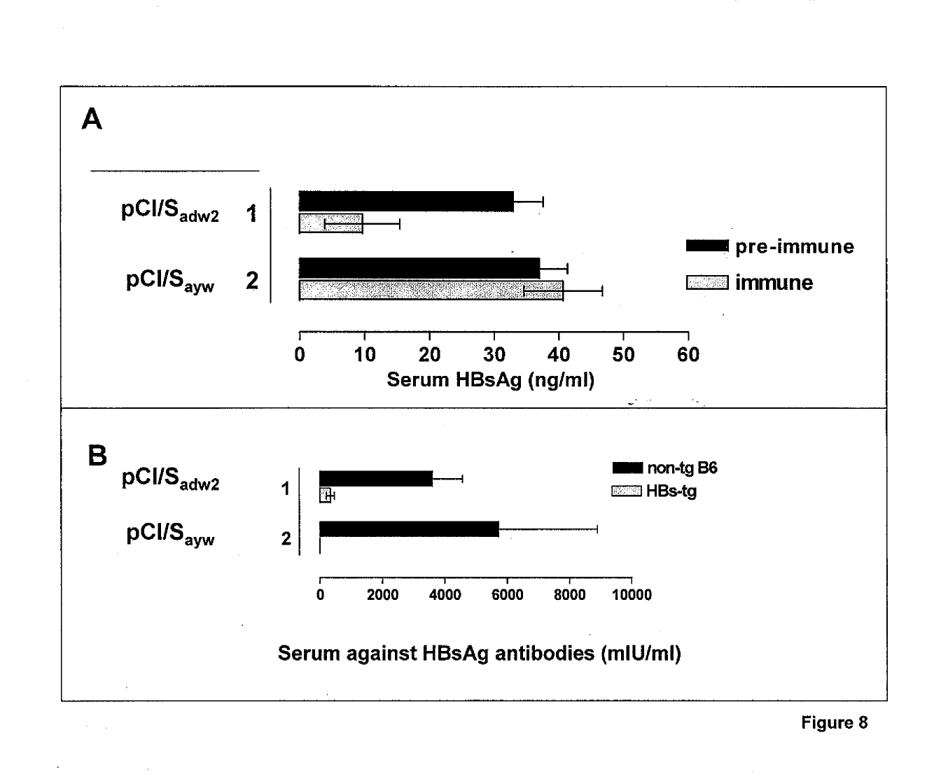

Untreated HBs-tg mice exhibit HBsAg serum levels of 30-50 ng/ml (FIG. 8A). Mice that developed cross-reactive CD8⁺ T-cell responses to epitope 1 after HBsAg_{adw2} immunisation exhibited reduced antigenaemia (with levels in the region of 5-15 ng/ml), whereas animals that had been immunised with HBsAg_{ayw}, which did not develop any HBsAg-specific CD8⁺ T-cell immunity, exhibited no change in antigenaemia levels (FIG. 8A). The partial control of antigenaemia therefore correlates with the occurrence of specific CD8⁺ T-cells in the immunised transgenic mice.

### Example 7

### Anti-HBsAg Serum Antibodies Occur in HBs_{ayw}-tg Mice that have been Immunised with HBsAg_{adw2}

In addition to T-cell immunity, the humoral anti-HBsAg immunity can play a role in the monitoring of antigenaemia. The occurrence of anti-HBsAg serum antibodies in vaccinated normal and transgenic mice was observed. Normal (non-transgenic) B6 mice and congenic HBs-tg B6 mice were immunised twice with pCL/S_{ayw} or pCL/S_{adw2} DNA vaccine. Their serum antibody titres, which were specific to HBsAg, were determined two weeks after the last immunisation using the ImxAUSAB test (Abbott) which determines HBsAg of different subtypes. While non-transgenic mice that had been immunised with pCL/S_{ayw} or pCL/S_{adw2} plasmid DNA developed high serum antibody levels to HBsAg, HBs-tg mice exhibited an anti-HBsAg serum antibody response only after immunisations with pCL/S_{adw2} (but not with pCL/S_{ayw}) plasmid DNA (FIG. 8B). Similar antibody responses were observed in mice immunised with HBsAg_{ayw} or HBsAg_{adw2} particles (data not shown). A subtype-specific ELISA (with HBsAg_{ayw} or HBsAg_{adw2} particle-coated plates) showed that in normal mice >95% of the antibody response produced by all vaccines is directed against the “a” determinant of HBsAg; in HBs-tg mice, >90% of the antibody response is directed against adw2-specific determinants (data not shown).

### Example 8

### Efficient Priming of Cross-Reactive K^{b}-Restricted CD8⁺ T-Cell Responses to HBsAg Epitope 1 in HBs-tg B6 Mice by Immunisation with HBsAg Protein Particles

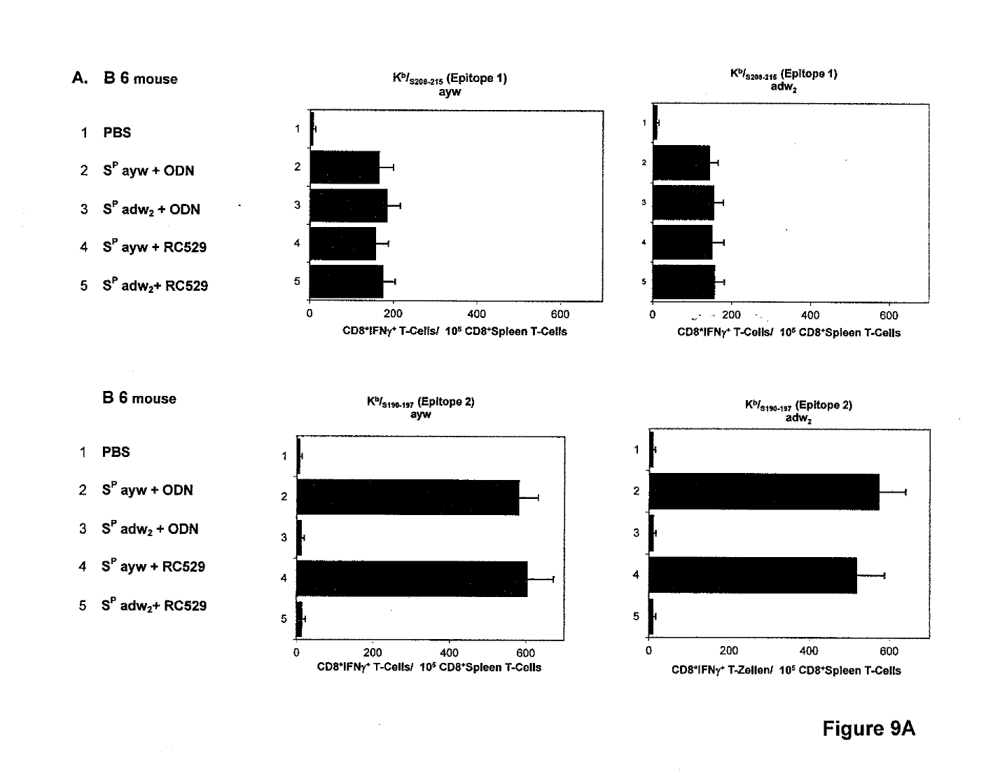

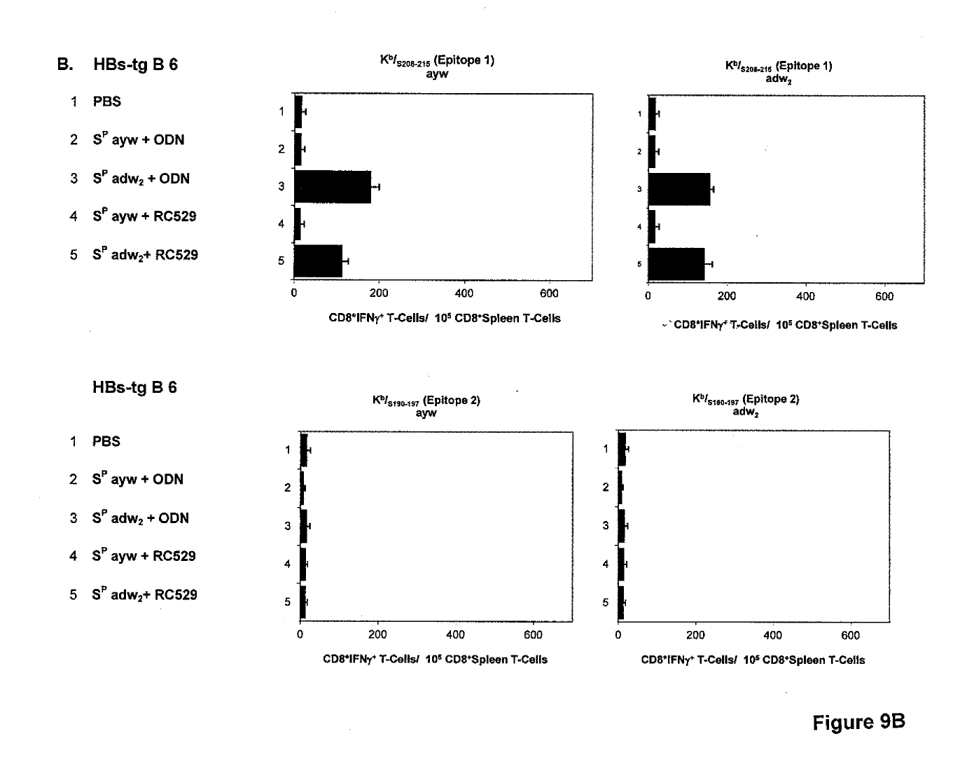

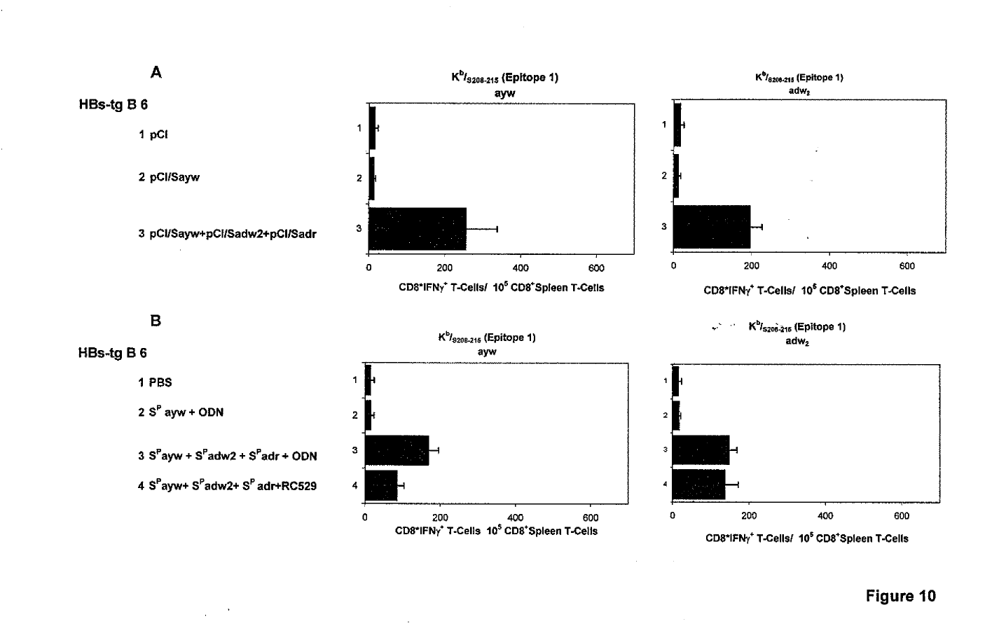

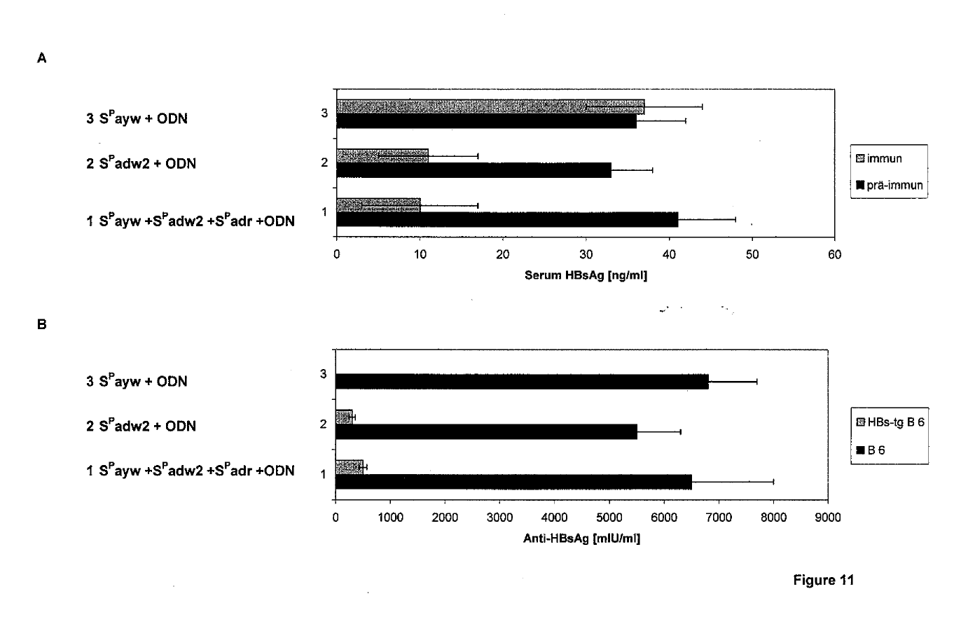

Immunisation of normal B6 mice with HBsAg protein particles of subtype ayw or adw2 results in a CD8⁺ T-cell-mediated immune response to the K^{b}-binding epitope 1 (S₂₀₈₋₂₁₅). FIG. 9A). It can thus be shown that irrespective of the nature of the vaccines (protein particles or DNA), epitopes having different sequences are able to prime cross-reactive T-cell responses. Analogously to the immunisations with DNA vaccines (FIG. 5), it has been found that vaccination of B6 mice with HBsAg protein particles of subtype ayw primes a CD8⁺ cell response to the HBsAg K^{b}-binding epitope 2 (S₁₉₀₋₁₉₇) but not vaccination with HBsAg protein particles of subtype adw2 (FIG. 9A).

HBs_{ayw}-tg mice were immunised with HBsAg protein particle vaccines corresponding to either subtype ayw or subtype adw2. Whereas no CD8⁺ T-cell response was generated after repeated immunisation with the HBsAg_{ayw} protein vaccine, immunisation with the heterologous HBsAg_{adw} protein antigen generated an HBsAg-specific CD8⁺ T-cell response to epitope 1 (FIG. 9B). It is thus demonstrated that a natural variant of HBsAg is able to break an existing tolerance by the priming of a cross-reactive T-cell response also by means of a protein subunit vaccination.

### Example 9

### Efficient Priming of Cross-Reactive K^{b}-Restricted CD8⁺ T-Cell Responses Towards HBsAg Epitope 1 in HBs-tg B6 Mice by Immunisation with Mixtures of Natural Variants of HbsAg

HBs_{ayw}-tg mice were immunised either with a DNA vaccine that coded for the three HBsAg subtypes ayw (pCl/S_{ayw}), adw₂ (pCl/S_{adw2}) and adr (pCl/S_{adr}) (FIG. 10A), as well as a HBsAg protein particle vaccine containing a mixture of subtypes ayw, adw₂ and adr (FIG. 10B). The mixture of natural variants of HBsAg primed cross-reactive K^{b}-restricted CD8⁺ T-cell responses to epitope 1 both after immunisation with DNA and with protein particles.

### Example 10

### Reduction of Antigenaemia in HBs-tg Mice after Immunisation with Mixtures of Natural Variants of HbsAg

In untreated HBs-tg mice, a serum level of 30-50 ng/ml is observed. Animals which, after immunisation with a heterologous HBsAg vaccine (HBsAg_{adw2}) or a mixture of natural HBsAg variants (HBsAg_{ayw}+HBsAg_{adw2}+HBsAg_{adr}(, develop a cross-reactive CD8⁺ T-cell response to epitope 1 exhibit reduced antigenaemia (with HBsAg levels of 5-17 ng/ml). In animals that were immunised solely with the homologous HBsAg_{ayw} and thus were unable to generate HBsAg-specific T-cell immunity, no change in the amount of antigen in the serum was observed. Immunisation with a mixture of natural variants of HBsAg can accordingly bring about a reduction in antigenaemia.

### Example 11

### Induction of Anti-HBsAg Serum Antibodies in HBs-tg Mice after Immunisation with Mixtures of Natural Variants of HbsAg

Normal B6 mice exhibit a marked antibody response after immunisation with HBsAg_{ayw}, HBsAg_{adw2}, HBsAg_{adr} (not shown) as well as with a mixture of the three subtypes.

The formation of HBsAg-specific serum antibodies in HBs-tg mice after immunisation was investigated. HBs-tg mice exhibited a serum antibody response only after immunisation with a mixture of natural HBsAg variants or with the heterologous subtype adw₂. No anti-HBsAg response was induced after immunisation with the homologous subtype ayw. A subtype-specific ELISA (microtitre plates coated with HBsAg_{ayw} and HBsAg_{adw2} protein particles) showed that in HBs-tg mice >90% of the HBsAg-specific antibody response is directed against adw2-specific determinants (data not shown).

### REFERENCES

1. Schirmbeck, R., Boehm, W., Fissolo, N., Melber, K., and Reimann, J., Different immunogenicity of H-2 K^{b}-restricted epitopes in natural variants of the hepatitis B surface antigen. Eur. J. Immunol. 2003. in press: xx-yy.
2. Ando, K.-I., Guidotti, L. G., Wirth, S., Ishikawa, T., Missale, G., Moriyama, T., Schreiber, R. D., Schlicht, H. J., Huang, S. N., and Chisari, F. V., Class I-restricted cytotoxic T lymphocytes are directly cytopathic for their target cells in vivo. J. Immunol. 1994. 152: 3245-3253.
3. S. Schaefer, M. Piontek, S. J. Ahn, A. Papendieck, Z. Janowicz, I. Timmermans, and G. Gellissen. 2002. Recombinant hepatitis B vaccines—disease characterization and vaccine production. in Hansenula polymorpha—Biology and Applications. G. Gellissen (ed.) pp 175-210, Wiley-VCH, Weinheim, Germany
4. Schirmbeck, R., Boehm, W., Ando, K.-I., Chisari, F. V., and Reimann, J., Nucleic acid vaccination primes hepatitis B surface antigen-specific cytotoxic T lymphocytes in nonresponder mice. J. Virol. 1995. 69: 5929-5934.
5. Boehm, W., Kuhröber, A., Paier, T., Mertens, T., Reimann, J., and Schirmbeck, R., DNA vector constructs that prime hepatitis B surface antigen-specific cytotoxic T lymphocyte and antibody responses in mice after intramuscular injection. J. Immunol. Methods 1996. 193: 29-40.
6. Trobonjaca, Z., Leithäuser, F., Moller, P., Schirmbeck, R., and Reimann, J., Activating immunity in the liver. I. Liver dendritic cells (but not hepatocytes) are potent activators of IFNγ release by liver NKT-cells. J. Immunol. 2001. 167: 1413-1422.
7. Trobonjaca, Z., Kroger, A., Stober, D., Leithauser, F., Moller, P., Hauser, H., Schirmbeck, R., and Reimann, J., Activating immunity in the liver. II. IFN-β attenuates NK cell-dependent liver injury triggered by liver NKT-cell activation. J. Immunol. 2002. 168: 3763-3770.
8. Buus, S., Stryhn, A., Winther, K., Kirkby, N., and Pedersen, L. O., Receptor-ligand interactions measured by an improved spun column chromatography technique. A high efficiency and high throughput size separation method. Biochim. Biophys. Acta 1995. 1243: 453-460.
9. Olsen, A. C., Pedersen, L. O., Hansen, A. S., Nissen, M. H., Olsen, M., Hansen, P. R., Holm, A., and Buus, S., A quantitative assay to measure the interaction between immunogenic peptides and purified class I major histocompatibility complex molecules. Eur. J. Immunol. 1994. 24: 385-392.
10. T. van-Hall, J. van-Bergen, P. A. van-Veelen, M. Kraakman, L. C. Heukamp, F. Koning, C. J. Melief, F. Ossendorp, and R. Offring a. 2000. Identification of a novel tumor-specific CTL epitope presented by RMA, EL-4, and MLB-2 lymphomas reveals their common origin. J. Immunol. 165:869-877

That which is claimed:

1. A method of preparing a medicament for the therapeutic treatment of hepatitis B, comprising the steps of:
(a) determining the HBV genotype with which a patient is infected; and
(b) providing a medicament comprising at least one HBsAg of an HBV genotype, a fragment thereof or a nucleic acid encoding HBsAg, the genotype thereof differing from the HBV genotype of the patient determined according to step (a).

2. The method of claim 1, wherein said determining step comprises determining the genotype by a PCR method.

3. The method of claim 1, wherein said determining step comprises determining the genotype by sequencing of the total HBV genome or at least the portion coding for the HBsAg and phylogenetic analysis.

4. The method of claim 1, wherein said determining step comprises determining the genotype by restriction fragment length polymorphism (RFLP).


SEQUENCE LISTING (7 sequences)

SEQ ID NO: 1 - protein, 226 aa, Hepatitis B virus
  HBsAg polypeptide of HBV subtype adw2/genotype A
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 2 - protein, 226 aa, Hepatitis B virus
  HBsAg polypeptide of HBV subtype ayw/genotype D
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 3 - protein, 8 aa, Hepatitis B virus
  HBV subtype adw2 HBsAg epitope 190-197
VWLSAIWM

SEQ ID NO: 4 - protein, 8 aa, Hepatitis B virus
  HBV subtype ayw HBsAg epitope 190-197
VWLSVIWM

SEQ ID NO: 5 - protein, 8 aa, Hepatitis B virus
  HBV subtype adw2 HBsAg epitope 208-215
IVSPFIPL

SEQ ID NO: 6 - protein, 8 aa, Hepatitis B virus
  HBV subtype ayw HBsAg epitope 208-215
ILSPFLPL

SEQ ID NO: 7 - protein, 226 aa, Hepatitis B virus
  HBsAg polypeptide of HBV subtype adr/genotype C
MENTTSGFLGPLLVLQAGFFLLTRILTIPQSLDSWWTSLNFLGGAPACPGQNSQSPTSNHSLTSCPPICPGYRWMCLRRFIIFLFTLLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI